(19) United States
(12) Patent Application Publication
(10) Pub. No.: US 2004/0151617 A1
(43) Pub. Date: Aug. 5, 2004

(54) Methods and apparatus for air sterilization

(76) Inventors: Michael F. Zanakis, Stuart, FL (US); Douglas C. Sampson, Palm City, FL (US)

Correspondence Address: KENYON & KENYON, 1500 K STREET, N.W., SUITE 700, WASHINGTON, DC, 20005, US

(21) Appl. No.: 10/355,165

(22) Filed: Jan. 31, 2003

(51) Int. Cl. A61L 9/00; B01J 19/00

(52) U.S. Cl. 422/4; 422/121; 422/122; 422/123; 422/186.05

(57) ABSTRACT

An air sterilization system comprising an adherent agent, a plurality of electrostatically charged non-conductive objects, and high-energy is disclosed. The system preferably includes an enclosed chamber, an adherent generator, and at least one high-energy source. The air produced by the sterilization system is breathable air.

A method in accordance with an embodiment of the invention is disclosed. The method includes flowing air through a channel, injecting adherent agent into the air in the channel, and irradiating at least a portion of an internal volume of the channel with a high-energy field. The channel is preferably filled with objects that acquire an electrostatic charge when irradiated with a microwave field. These objects preferably maintain at least a portion of their charge when substantially enveloped in a vapor of adherent agent.

### FIELD OF THE INVENTION

The invention relates to methods and apparatus for producing sterilized breathable air.

### BACKGROUND

Airborne particles that constitute, contain or have been released from living organisms are present in most environments. Such airborne microorganisms typically include viruses, bacteria and spores and may be pathogenic. Thus in certain environments—e.g., in hospitals, nursing homes, office buildings or airplanes—it may be desirable to eliminate essentially all potentially pathogenic particles from the air, i.e., sterilize it. This elimination is difficult because in part such particles range in size from approximately 0.005 microns in diameter (e.g., a virus) to 100 or more microns in diameter (e.g., spores, can be 2 to 200). Air purification systems, which are primarily based on filtration techniques, are not capable of achieving this result and thus cannot be termed air sterilizers.

By far, the most widely studied and applied air purification system is the barrier or filter technique employed in air ventilation systems. Generally, three types of filters exist: pre-filters, HEPA (High Efficiency Particulate Air) filters, and ULPA (Ultra Low Penetration Air) filters. Typically, a pre-filter can filter air with a 70-90% efficiency. This means that 70-90% of all airborne particulates under a given size will be captured by the filter and thus eliminated from the air. HEPA and ULPA filters are said to filter air with about 95-97% efficiency, and can produce a barrier to particles of 1 micron in diameter or slightly smaller. However, filters with very small barriers typically require more energy to operate and are thus more costly.

Furthermore, while some conventional state-of-the-art HEPA filters have reported efficiency ratings of 99.7% for removing particulates above 0.3 microns in diameter, such efficiencies are determined under ideal laboratory conditions, and actual efficiencies upon installation and use usually fall short of the reported efficiencies.

Many outside factors affect filter efficiency so that often the actual efficiency upon installment and use is lower than the stated, lab-determined, efficiency. For example, filter performance is greatly affected by alterations in filter installation techniques. Improper fitting of frames, ductwork, and non-uniformity of airflow all have a major impact on a filter's performance. Other physical factors that can impact a filter's efficiency, include operational airflows outside the operating limits of a given system, or small changes in temperature and humidity.

Another factor often overlooked by HEPA filter designers is that bacteria and viruses are dynamic living entities. Unlike inanimate particulate matter, they tend to avoid being attached to a substrate that does not provide nutrition.

### SUMMARY OF THE INVENTION

An air sterilization system comprising an adherent agent, a plurality of electrostatically charged non-conductive objects, and high-energy to sterilize air is disclosed. The system preferably includes an enclosed chamber having an inlet opening and an outlet opening and a channel extending between the two openings to allow air to flow into and out of the channel, respectively. The plurality of electrostatically charged non-conductive objects are preferably located in the channel. An adherent generator that injects adherent agent into the air to be sterilized is preferably coupled to the channel. The system preferably includes at least one high-energy source to irradiate at least a portion of the channel.

A method in accordance with an embodiment of the invention includes allowing air to flow through the channel, injecting adherent agent into the air in the channel (e.g., in the form of a vapor), and irradiating at least a portion of an internal volume of the channel with a high-energy field. Preferably, the channel is substantially filled with objects that acquire an electrostatic charge when exposed to the high-energy field, such as a microwave field. These objects preferably maintain at least a portion of their charge when substantially enveloped in adherent agent. In one embodiment, the objects are spheres. Each sphere has a hollow internal chamber defined by an internal wall that forms a non-permeable barrier to the adherent agent.

The various features of the invention will best be appreciated by simultaneous reference to the following description and accompanying drawings. In the accompanying drawings, like numerals indicate like elements.

### BRIEF DESCRIPTION OF THE DRAWINGS

FIG. 1 is an exploded view of an apparatus in accordance with an embodiment of the invention.

FIG. 2 illustrates an alternate embodiment of an apparatus in accordance with an embodiment of the invention.

FIG. 3 illustrates a cutaway top view of an airflow chamber in accordance with an embodiment of the invention.

FIG. 4 illustrates a cutaway cross-sectional view of one embodiment of a non-conductive object in accordance with an embodiment of the invention.

FIG. 5 is a flowchart depicting a method in accordance with an embodiment of the invention.

FIG. 6 depicts a cutaway view of a system used to perform laboratory experiments to test the efficacy of air sterilization in accordance with an embodiment of the invention.

FIG. 7 is an illustration of a tool, in accordance with an embodiment of the invention, used to manufacture a hollow impression in one hemisphere of a sphere.

FIG. 8 illustrates a cutaway cross-sectional view of another embodiment of a non-conductive object in accordance with an embodiment of the invention.

### DETAILED DESCRIPTION OF AN EMBODIMENT OF THE INVENTION

The present invention relates to a method and apparatus for producing sterilized breathable air. By sterilized it is meant that more than 95%, and preferably 100%, of microorganisms in the breathable air are neutralized or destroyed. Sterility, for purposes of this invention, is measured using an impaction-capture method for microbial sampling. According to this test method, one of three types of microbes is injected into the air inlet of the air sterilization system disclosed herein. Microbes may be either: 1) dried Brevi bacterium linens; 2) dried Serratia marscesens; or 3) Penicillium. An agar-coated petri dish is placed at an air outlet of the system. Any microbes exiting the system randomly hit and attach (and subsequently grow, or not) on the dish. Dishes are cultured from 24 to 48 hours. Multiple samplings are taken and compared to control situations. One-hundred percent sterility may be defined as “the absolute lack of growth in the petri dish of the specific type of microbe injected into the system.” Other measures of sterility may be defined with reference to a control situation. It must be noted that the definition of sterility does not exclude all growth on the petri dish. During any sterility assessment period, the petri dish may become contaminated with microbes other than those injected into the system in accordance with an embodiment of the invention. Such contamination may be avoided if the entire system is tested in a sterile environment, where the only microbial contamination was from those microbes purposefully injected into the system for the purpose of testing sterility. A summary of experimental results of measurements using the impaction-capture method in a non-sterile test environment is included herein.

Airborne microorganisms, including pathogens, can be neutralized or destroyed, to effect sterilization, if they are made to absorb enough high-energy to disrupt the molecular processes necessary for life and subsequent pathogenicity. The amount of high-energy absorption required, however, is dependent on the size of the microorganism and under normal atmospheric conditions, airborne pathogens are of such a small size that they absorb little energy. It has been found that if airborne microorganisms are made to present a large physical profile to the applied incident energy, more energy is absorbed by the resultant larger structure. It has further been found that such a large profile can be achieved by allowing the airborne pathogen to become adsorbed to a larger carrier material or adherent agent that can be introduced into the breathable air to be sterilized. A preferred example of an adherent agent is water vapor, but such adherent agents may include any other gaseous or vaporous substance upon which the microorganisms can become adsorbed or that enables the airborne microorganisms to agglomerate. Other examples of carrier materials or adherent agents include Xenon gas, alcohol, and colloidal carbon. This list is not meant to be exclusive. Provided the microorganism-adherent complex is mobile enough to be conveyed along an air path, then the air to be sterilized can take a course through a field of high-energy irradiation. As the microorganism-adherent complex passes through the field, the applied high-energy, such as microwave, may be absorbed by the complex causing destruction or neutralization of the microorganism. Microorganisms may also be destroyed or neutralized as a result of the indirect absorption of heat. The adherent agent can then be removed from the air path by such methods as dehumidification in circumstances where water vapor is used as the carrier. The condensate can be neutralized for humidification as well, thereby creating a closed-loop for water in the system. A barrier filter can also be placed in the air path to remove particulate material.

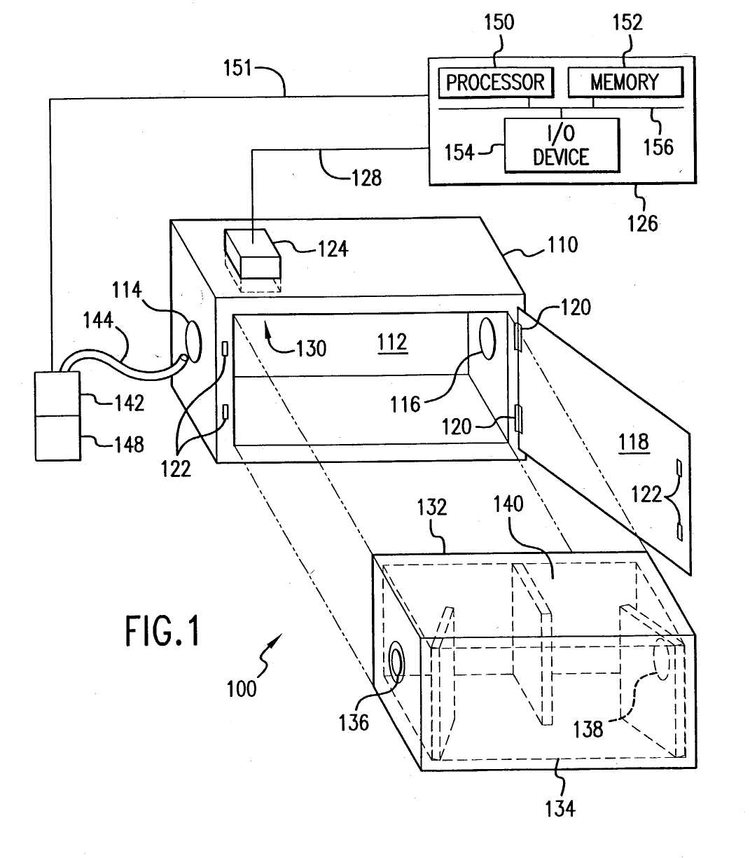

FIG. 1 is an exploded view of an apparatus 100 in accordance with an embodiment of the invention. The apparatus includes a sterilization chamber 110. The sterilization chamber, 110 illustrated in the embodiment shown in FIG. 1 is rectangular in shape and thus includes an internal volume 112 which is bounded by six internal walls. The sterilization chamber 110 may take on other forms (e.g., square, triangular, cylindrical), and may thus have internal volumes bounded by more or less than six internal walls without departing from the scope of the invention. The internal walls of the sterilization chamber 110 separate a volume of space outside of the walls of the sterilization chamber 110 from an internal volume of space 112 within the sterilization chamber 110. The sterilization chamber 110 may shield the volume of space outside the walls of the sterilization chamber 110 from a high-energy field that may be irradiated within the internal volume 112 of the sterilization chamber 110.

The sterilization chamber 110 includes an air inlet opening 114 and an air outlet opening 116 to allow entrance and exit, respectively, of air to and from the internal volume 112 of the sterilization chamber 110. While illustrated as circular openings, air inlet and outlet openings 114, 116 may take on any form and may be any size which allows for a volume of air to pass through the apparatus 100 at a rate sufficient for the destruction or neutralization of any microorganisms suspended in the airflow. Air inlet and outlet openings 114, 116 may be operatively covered or baffled (not shown) such that air may pass through the cover or baffle while any high-energy present within the internal volume 112 of the sterilization chamber 110 is substantially contained therein. The types of coverings or baffles used may be dependent upon the type of high-energy which is to be contained within the internal volume 112 of the sterilization chamber 110 and those of skill in the art will be able to determine suitable materials and form factors without undue experimentation. For example, if microwave energy is used to illuminate the internal volume 112 of the sterilization chamber 110, then a metal wire mesh may be used to cover the openings 114, 116, thus allowing air to pass through the openings in the metal wire mesh but reflecting any microwave energy that could potentially escape through the openings 114, 116.

One of the walls of the sterilization chamber 110 may be removably secured to the sterilization chamber 110. The removably secured wall 118 may be removed to provide access to the internal volume 112 of the sterilization chamber 110. Fasteners (not shown) may secure the removably secured wall 118. Fasteners such as screws, clamps, magnetic seals, quarter or half-turn securing devices, among others may be used. Any fasteners known to those of skill in the art may be used. Alternatively, a portion of one of the walls of the sterilization chamber may be adapted to provide access, via an access door or panel, to the internal volume 112 of the sterilization chamber. In the embodiment shown in FIG. 1, the removably secured wall 118 is a door that is secured on one edge by one or more hinges 120 and secured on other edges by one or more latching mechanisms 122. While latching mechanisms 122 are shown on the edge of the removably secured wall 118 opposite to the edge having hinges 120, other locations may be chosen without departing from the scope of the invention.

The apparatus includes at least one high-energy source 124. In the embodiment of FIG. 1, the high-energy source 124 emits its high-energy into substantially the entire internal volume 112 of the sterilization chamber 110. The high-energy source 124 may be, for example, a microwave source or an ultraviolet light source. This list is intended to be exemplary and not exclusive.

The high-energy source 124 may be controlled by control circuitry 126. Control circuitry 126 may be integrated into the sterilization chamber 110 or may be located remotely from the sterilization chamber 110. Interface between the high-energy source 124 and the control circuitry 126 may be made via an interface 128, which may be a wired, a wireless, an optical, or any other interface known to those of skill in the art. The control circuitry 126 may include a processor 150, a memory 152, and an input/output device 154 that are all coupled to a communications bus 156. The communications bus 156 may be coupled to the interface 128.

The high-energy source 124 may emit its energy into the internal volume 112 of the sterilization chamber 110 through an opening 130 in a wall of the sterilization chamber 110. The high-energy source 124 may be oriented to direct its high-energy in any direction into the internal volume 112 of the sterilization chamber 110. In one embodiment, the orientation provides dispersal of high-energy throughout substantially the entire internal volume 112 of the sterilization chamber 110. A device (not shown) (e.g., a waveguide, antenna, reflector, or lens) may be used in cooperation with the high-energy source 124 to direct the high-energy emitted from the high-energy source 124 into the internal volume 112 of the sterilization chamber 110. Multiple high-energy sources may be used without departing from the scope of the invention. In one embodiment, one high-energy source is used. In one embodiment, the high-energy source 124 is a microwave magnetron. The microwave magnetron operates at frequencies of either approximately 915 MHz or 2,450 MHz, corresponding to wavelengths of approximately 132.8 cm and 112 cm, respectively. However, microwave energy of various frequencies can be used either separately or in concert, as one wavelength may be more optimal than another for purposes of destroying or neutralizing certain types of microorganisms. The high-energy source or sources may be placed and/or focused in one area of the sterilization chamber 110, or may be placed and/or focused substantially along the path of air travel through an airflow chamber 132. The high-energy source 124 may emit energy directly into the airflow chamber 132 or may be designed to emit energy that penetrates the airflow chamber 132 if, for example, the airflow chamber is to be placed inside of the sterilization chamber 110 as a removable unit. Specific placement configurations may be chosen to maximize the efficiency of the system for destruction and/or neutralization of microorganisms, and at the same time to reduce air obstructions by any objects positioned in the airflow chamber. Reduction of airflow obstructions increases airflow and subsequent energy efficiency of the system.

The apparatus 100 may include an airflow chamber 132. The airflow chamber 132 may be removable from the sterilization chamber 110 or it may be constructed as an integral part of the sterilization chamber 110. Removability supports the reuse of the airflow chamber (after it has been cleaned). Removability also supports the replacement of the airflow chamber 132 if, for example, the airflow chamber 132 is considered a disposable portion of the apparatus 100. Cleaning or replacement of the airflow chamber 132, may be required on a periodic basis as destroyed or neutralized microorganisms collect within the interior volume of the airflow chamber 132. Removability of the airflow chamber 132 also supports the reuse or replacement of any objects that may be placed into the interior volume of the airflow chamber 132. Such reuse or replacement may be effected via a cover or removable wall (not shown) on the airflow chamber 132.

The airflow chamber 132 may be manufactured or constructed from a material that is transparent or substantially transparent to the high-energy emitted from the high-energy source 124. For example, if the high-energy source was a microwave energy source, the chamber may be constructed of plastic (such as high-density polyethylene), paper, expanded polystyrene, or glass. This list is, of course, not meant to be exclusive. In a preferred embodiment, expanded open-cell polystyrene is used.

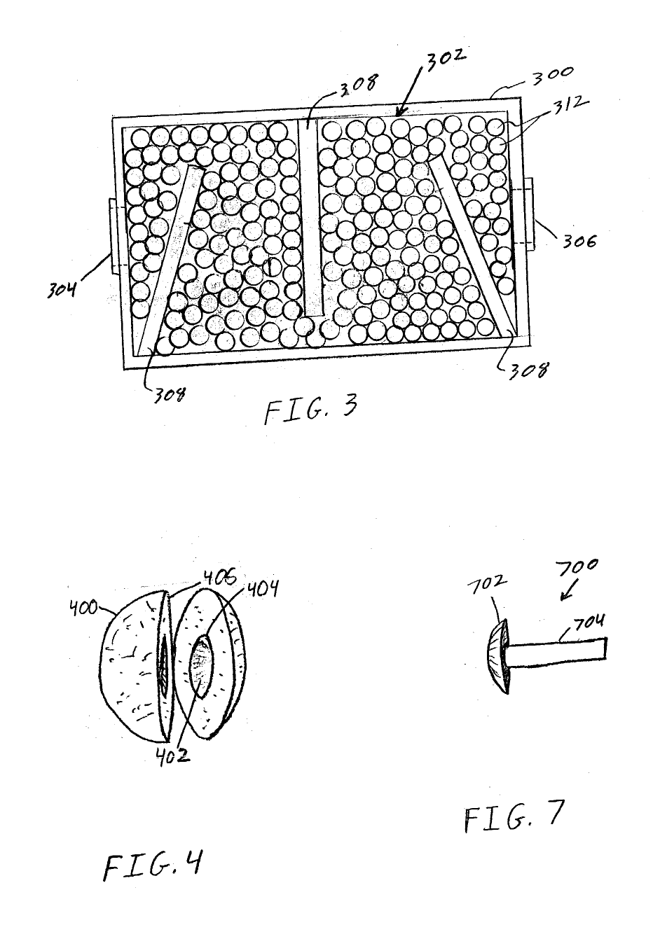

The airflow chamber 132 is comprised of an enclosed chamber 134 having an inlet opening 136 and an outlet opening 138 and a channel 140 extending therebetween. The airflow chamber 132 fits within the internal volume 112 of the sterilization chamber 110, however, it need not occupy the entirety of the internal volume 112. Inlet opening 136 and outlet opening 138 make a substantially airtight seal with air inlet opening 114 and air outlet opening 116, respectively. In one embodiment, a friction fit between the walls of the sterilization chamber 110 and the airflow chamber 132 establishes the substantially airtight seal. The degree of airtight seal existing between the airflow chamber 132 and the sterilization chamber 110 may be increased or decreased according to the application for which the apparatus is used. Those of skill in the art may increase or decrease the degree of airtight seal without departing from the scope of the invention. The airflow chamber is further described with reference to FIG. 3, below. In an alternate embodiment (not shown), the airflow chamber itself may incorporate one or more high-energy sources; in which case the high-energy source 124 of the sterilization chamber 110 may not be used or may not be present.

The apparatus 100 may also include an adherent generator 142 that may be operatively coupled to the air inlet opening 114 of the sterilization chamber 110, the inlet opening 136 of the airflow chamber 132, and/or the channel 140 of the air flow chamber 132. The adherent generator 142 may generate an aggregation of fine drops of an adherent agent that may be suspended in the air. The fine drops of adherent agent, (i.e., the vapor of adherent agent) may act as a carrier for microorganisms suspended in the breathable air passing into the sterilization chamber 110. In one embodiment, the adherent agent may be any agent that, when converted to a vapor, possesses a net static charge such that the microorganism-adherent complex (i.e., at least one microorganism in combination with at least one particle of the adherent agent vapor) becomes a dipole.

The adherent generator 142 may be an atomizer, a mister, a vaporizer, a humidifier, or any device that produces a vaporous adherent agent. In one embodiment, the adherent agent is water and the adherent generator is a vaporizer that uses an ultrasonic generator to produce water vapor. In one embodiment, the water vapor is expelled from the vaporizer and is conducted to the air inlet opening 114 by a vaporizer conduit 144. In the embodiment illustrated in FIG. 1, the vapor produced by the vapor generator 142 passes through the vaporizer conduit 144 and is pulled into the air inlet opening 114, and thus into the channel 140 of the airflow chamber 132, by virtue of air that is also being pulled, or pushed, into the air inlet opening 114. In this embodiment, an output port 146 of the vaporizer conduit 144 lies adjacent to the air inlet opening 114. Other configurations or apparatus may be used to deliver vapor to the channel 140 without departing from the scope of the invention.

Air may be pulled or pushed into the air inlet opening 114 by an air handling system into which the apparatus 100 is installed. The air handling system may use an airflow apparatus (e.g., any suitable type of fan or blower or the like know to those of skill in the art) to move air through the air handling system. In another embodiment, the apparatus 100 may include its own auxiliary airflow apparatus, which may be used instead of or in addition to the airflow apparatus of the air handling system. Such an auxiliary airflow apparatus may be desirable if, for example, the air resistance of air passing through the apparatus 100 is of a magnitude that could not be overcome by the normal rate of air flowing through the air handling system. Those of skill in the art will readily understand the calculations necessary to determine whether such an auxiliary airflow apparatus is desirable.

The air that may be pulled or pushed into the air inlet opening 114 may be breathable air that has been completely or partially recirculated within the air handling system. In other words, the air handling system may receive, through return vents in the system, a quantity of breathable air that exists, for example, in a room or in an airline cabin. That quantity of breathable air, possibly along with breathable air from other sources, can then be pulled or pushed through the apparatus 100 and undergo sterilization. The sterilized breathable air may then be returned to the room from which it came, or to other rooms.

The adherent generator 142 may be operatively coupled to a reservoir 148 to hold a quantity of adherent agent. The adherent generator 142 may be controlled by control circuitry 126 or other control circuitry (not shown) in order to facilitate that starting or stopping of the generation of adherent vapor, or the amount or rate of generation of adherent vapor. Interface between the control circuitry 126 and adherent generator 142 may be made via any interface known to those of skill in the art. A wired interface 151 is illustrated in FIG. 1.

As more fully explained below, the vapor produced by the vapor generator 142 as well as the air passing through the channel 140 traverses the length of the channel 140 and exits the sterilization chamber 110 at air outlet opening 116.

The apparatus may also include an air circulator (not show), which may be, for example, a fan. The air circulator may be a part of an air handling system, such as a heating ventilation and air conditioning (“HVAC”) system within which the apparatus 100 is installed, or it may be incorporated into the apparatus 100. The air circulator may push or pull the air through the airflow chamber 132. The rate of air being pushed or pulled through the airflow chamber 132 may be chosen to maximize energy efficiency. The apparatus may also include a barrier filter.

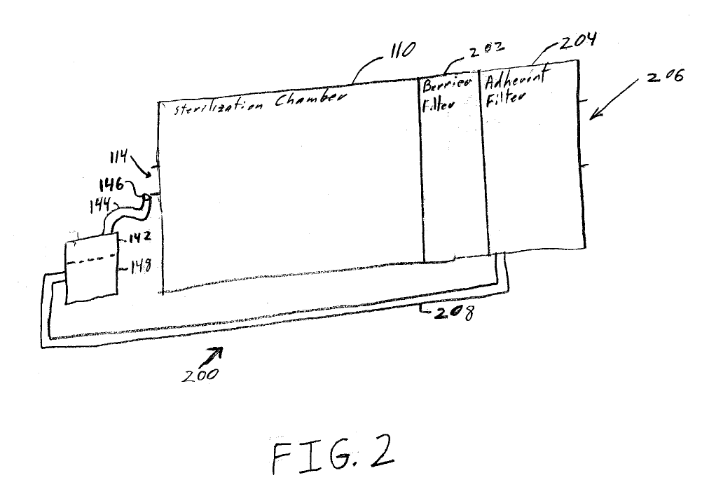

FIG. 2 illustrates an alternate embodiment of an apparatus in accordance with an embodiment of the invention. In FIG. 2, the apparatus 200 includes a vaporizer 142, a vaporizer reservoir 148, a vaporizer conduit 144, a sterilization chamber 110, an airflow chamber 134 (inserted within the sterilization chamber 110 and not shown for ease of illustration), a barrier filter 202, an adherent filter 204, and a condensate conduit 208. Air enters the apparatus 200 through an air inlet opening 114 in the sterilization chamber 110. Vapor from the adherent generator is pulled into the sterilization chamber 110 and flows through the channel 140 in the airflow chamber 134 (not shown). High-energy irradiates the breathable air flowing through the channel 140. Particulate may be trapped within the channel 140 and/or on any objects that may have been placed within the channel 140. Any particulate in the vapor and air combination exiting the air sterilization chamber 110 via air outlet opening 116 (not shown) that has not been trapped within the airflow chamber 134 may exit the sterilization chamber via air outlet opening 116 (not shown) and be trapped by a barrier filter 202. The barrier filter 202 may be an active or passive filter. It may be designed to be removable, replaceable, and/or cleanable. The barrier filter 202 may be, for example, a pre-filter, HEPA, or ULPA type filter. It may include, for example, a removable canister, bag, or sheet. Of course, the above lists are exemplary and not meant to be limiting.

The vapor and air combination passing through the barrier filter 202 may then pass through an adherent filter 204, which may separate some quantity of the adherent present in vapor form from the vapor and air combination exiting the sterilization chamber 110. After separation, the sterilized breathable air may exit the adherent filter 204 from filter outlet opening 206 while the separated vapor may be condensed and returned to the reservoir 148. In an embodiment, the adherent filter 204 is a dehumidifier that condenses water vapor into liquid water. The condensate may itself be purified and/or filtered before being returned to reservoir 148 for regeneration into vapor. Return may be accomplished via a return conduit 208 coupling the adherent filter 204 to the reservoir 148. In an alternate embodiment, the adherent filter 204 need not be used.

Placement of the barrier filter 202 may be altered without departing from the scope of the invention. For example, while the barrier filter 202 of FIG. 2 is illustrated as preceding the adherent filter 204, the positions of the two filters may be reversed without departing from the scope of the invention. Furthermore, the barrier filter 202 may precede the sterilization chamber 110, without departing from the scope of the invention. Furthermore, the barrier filter 202 may be incorporated within the sterilization chamber 110, or within the airflow chamber 132. In an alternate embodiment, any objects used in the airflow chamber 132 may incorporate filtration material, such as activated carbon, in order to remove particulate from the air as the air passes through the airflow chamber 132. One consideration in adding filtration material into the airflow chamber is that the material should preferably be transparent or substantially transparent to the high-energy irradiating the airflow chamber. If, for example, the high-energy used was microwave energy, then two dissimilar materials adjacent to each other may generate heat, which could adversely affect the mechanical structure of the airflow chamber.

FIG. 3 illustrates a cutaway top view of an airflow chamber 300 (similar to 132 FIG. 1) in accordance with an embodiment of the invention. The airflow chamber 300 may be designed so that the path of the flow of air through the chamber is an indirect path from an inlet opening 304 (similar to 136 FIG. 1) to an outlet opening 306 (similar to 138 FIG. 1). The more indirect the path the more time a given microorganism traveling at a given flow rate has to be irradiated with high-energy that is irradiating the internal volume of the airflow chamber 300. To produce the indirect course, a channel 302 (similar to 140 FIG. 1) extending between the inlet opening 304 and the outlet opening 306 of the airflow chamber 300 may be, for example, a tortuous channel, or may be comprised of a regular or irregular set of bends and/or turns. A tortuous channel may be established by substantially filling the channel 302 with a collection of objects 312, such as spheres or plates, such that the air traveling through the channel is forced through the interstices between the objects.

In the embodiment of FIG. 3, the channel 302 is established by the incorporation of internal walls 308 extending from a top inner surface of the airflow chamber 300 to a bottom inner surface of the airflow chamber. In the embodiment of FIG. 3, each internal wall 308 may be bonded, pressed, friction fit, or otherwise attached or made an integral part of the airflow chamber on three of its four edges. The gap between the edge that is not in contact with the airflow chamber and the nearest adjacent wall of the airflow chamber forms a path through which air may travel through the channel 302. Other configurations of internal walls may also be used. In the embodiment of FIG. 3, the internal walls 308 are arranged in a non-parallel manner to maximize the flow of air through the airflow chamber and to minimize any locations where airflow may become stagnant. The internal walls 308 may be manufactured from the same material as used to manufacture the airflow chamber 300. One external wall of the airflow chamber 300 may serve as a lid for access to the internal volume of the airflow chamber 300. Alternatively, a portion of an external wall may include an opening for access to the internal volume of the airflow chamber 300. Alternatively, access to the internal volume of the airflow chamber 300 may be through the air inlet or outlet openings 304, 306.

In the embodiment illustrated in FIG. 3, the channel 302 is substantially filled with a plurality of objects 312. The objects 312 may be electrically non-conductive. The objects 312 may have the characteristic of not interacting adversely or interfering with the high-energy irradiation. The objects 312 may also have the characteristic of being transparent or substantially transparent to the high-energy field. The objects 312 may be manufactured from the same material as used to manufacture the airflow chamber 300. The air flowing through the channel 302 may be forced to pass around the objects placed within the channel, thus increasing the tortuousness of the path which must be taken by the air passing through the channel 302. In the embodiment of FIG. 3, the objects 312 are spheres. In other embodiments, the objects may be other regularly or irregularly shaped objects (such as plates or peanut shapes). Furthermore, objects having dissimilar shapes and/or sizes may be simultaneously placed within the channel without departing from the scope of the invention. Effectively, the addition of the objects 312 establishes “micro-channels” formed between the surfaces of each of the plurality of objects 312 inserted in the channel 302. Thus, the objects 312 further increase an effective path that is traversed by the air and/or air adherent mixture, as it flows through the channel 302. The path traveled from inlet opening 304 to outlet opening 306 is increased because the air is forced to maneuver around the walls 308 as well as travel around the surfaces of each of the plurality of objects 312 inserted in the channel 302.

In one embodiment, the objects 312 are electrically non-conductive and have a characteristic of acquiring an electrostatic charge as a result of exposure to a high-energy field, such as a microwave field. Consequently, a charged airflow chamber can act as a filter and hold particulate matter as well.

An important consideration in HVAC systems is air flow efficiency. Typically, the pressure drop across a filter is a crucial factor in determining an HVAC's efficiency. The lower the pressure drop, the more efficiently air can pass through the filter. However, typical barrier filters require air to pass through filter material so that the filter material can trap desired particulate. The higher the density of filter material the smaller the particulate that may be trapped. However, higher density filter material serves to decrease the HVAC's operating efficiency. On the other hand, a filter material, such as statically charged expanded polystyrene spheres allow for greater air flow efficiency, because the collection of spheres is less dense, and offers less of a barrier, than conventional barrier filters. The greater air flow efficiency results because air travels along a tortuous route through the micro-channels in the relatively large interstices between spheres; the particulate to be trapped is trapped by electrostatic attraction, snaring, or impaction of the particulate to the surface of the sphere.

In one embodiment of the invention, particulate matter, which may include microorganisms, whether or not adhered to any adherent vapor present in the system, attach to the objects 312 as the particulate collides with or passes by the objects 312. Attachment may occur due to electrostatic charge build-up on a surface of the object 312 or may occur due to surface features of the object trapping the particulate. The attachment of the particulate to the objects prolongs their duration in the airflow chamber 300 and thus prolongs their exposure to the destructive or neutralizing effects of high-energy irradiation.

The objects 312 may have various shapes and may be made from various materials. In the embodiment of FIG. 3, the objects 312 may be manufactured from the same material used to manufacture the airflow chamber 300 itself.

FIG. 4 illustrates a cutaway cross-sectional view of one embodiment of a non-conductive object 400 (similar to 312 FIG. 3), in this case, a cross sectional view of a one-inch diameter hollow sphere. In a preferred embodiment, the non-conductive objects 400 are one-inch diameter spheres made of open-cell expanded polystyrene or Styrofoam®. The spheres may have a hollow inner chamber 402. The interior volume of the non-conductive object 400 is substantially isolated from the outside surface 406 of the non-conductive object 400 by a substantially non-permeable layer 404 adjacent to the hollow inner chamber 402 of the sphere.

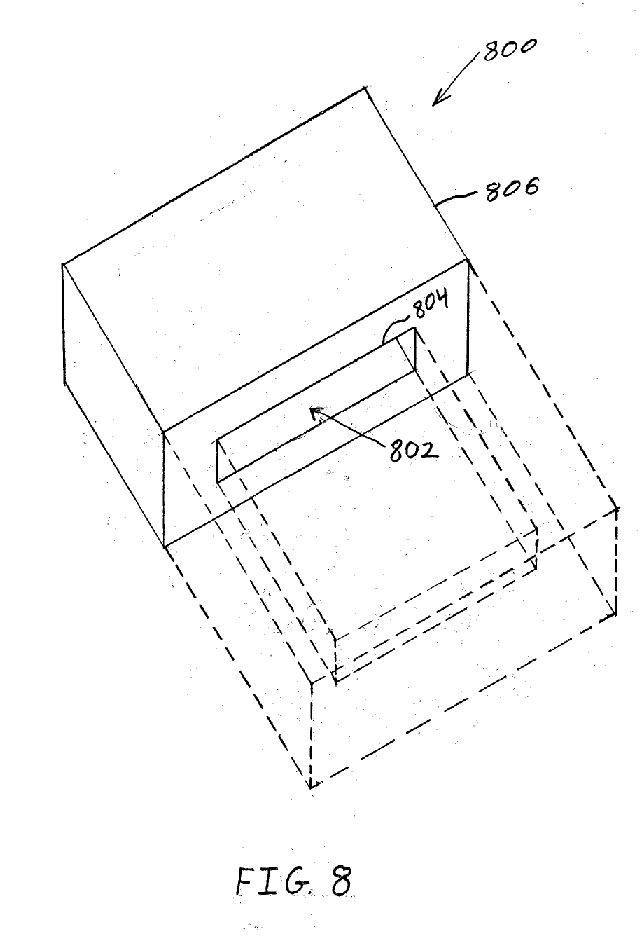

FIG. 8 illustrates a cutaway cross-sectional view of another embodiment of a non-conductive object 800. In the embodiment of FIG. 8, the non-conductive object is a rectangular plate, although other shapes, such as square or triangular, would be acceptable. The plate 800 may be manufactured from open-cell expanded polystyrene. The plate 800 may be formed to include a hollow inner chamber 802. Again, the interior volume of the plate 800 is substantially isolated from the outside surface 806 of the plate by a substantially non-permeable layer 804 adjacent to the hollow inner chamber 802 of the plate. A plurality of plates may be inserted into the airflow chamber such that a channel, comprised of a regular or irregular set of bends and/or turns, is established between the gaps and/or edges of adjacent plates from an air inlet to an air outlet of an airflow chamber. Nothing herein is meant to limit the configuration of any plates inserted into the airflow chamber, for example, plates may be inserted such that they are parallel to the airflow through the airflow chamber.

Expanded polystyrene that is irradiated with microwave energy develops an electrostatic charge. The electrostatic charge dissipates with time (several minutes) if the microwave energy is discontinued. The use of a conductive vapor, such as water vapor, that would coat a surface of an electrostatically charged expanded polystyrene object (such as a sphere or plate) could reduce the electrostatic charge build-up on the exterior surface of the object. However, the charging of an object made of expanded polystyrene can be maintained under vaporous conditions if the object includes a hollow center, where the hollow center (which, for a sphere may comprise approximately 50% of the sphere's total volume) is sealed from the remainder of the object by collapse of the expanded polystyrene cells. The collapse of the cells on the wall of the hollow surface creates a substantially non-permeable layer adjacent to the hollow inner chamber (e.g., the substantially non-penneable layer 404 adjacent to the hollow inner chamber 402 of the sphere 400) and thus creates a substantially airtight pocket within the object. In another embodiment, an object made of open-cell expanded polystyrene having a core made of solid polystyrene may also maintain a charge in the presence of a conductive vapor. It will be understood that other, either regularly or irregularly shaped objects, having a uniform density, or having cores that are hollow, or cores that are substantially filled may be used. These other, either regularly or irregularly shaped objects, may be made of open-cell expanded polystyrene or other material or materials having substantially the same physical and electrical characteristics.

In operation, using a sphere as an example, the entire sphere, including the sphere's hollow inner surface, may be statically charged in a microwave field. As incoming vapor dissipates the charge build-up on the surface of the sphere, but charge on the sphere's hollow inner surface substantially maintains its charge. Charge is substantially maintained because the interior is substantially impermeable to the vapor, and because the vapor does not easily penetrate the outer surface 406 of the sphere. The substantial maintenance of charge allows the hollow expanded polystyrene sphere to electrostatically attract and electrostatically hold any passing microorganism-adherent complex passing by or impacting the exterior surface 406 of the sphere 400. In the absence of microwave irradiation, of course, the charge on the inner surface of the hollow sphere dissipates.

FIG. 7 is an illustration of a tool, in accordance with an embodiment of the invention, used to manufacture a hollow impression in one hemisphere of a sphere. The tool 700 may be used to form impressions in polystyrene and other materials. The tool 700 is comprised of a metallic hemisphere 702 having a convex surface whose outside radius is equal to the inside radius of the impression that is to be formed in the hemisphere of, for example, a hemispherical portion of a polystyrene sphere. A handle 704 extends from the tool 700 in a direction opposite from the convex surface of the tool 700.

One method of manufacturing an expanded polystyrene sphere having a hollow inner chamber is to cut a solid sphere of polystyrene into approximately equal first and second halves. A tool 700, as described above, is heated to a temperature sufficient to melt the polystyrene. The tool is then pressed into the previously cut first half of sphere to form a concave impression. The surface of the concave impression is in parallel alignment with the exterior surface of the first half of sphere. The surface of the concave impression is comprised of melted polystyrene, which forms a substantially non-permeable surface to many liquids and gasses. For example, the surface manufactured in this way will be substantially non-permeable to water vapor. The same process is repeated for the second half of the sphere. The first and second halves are then bonded together using a melting agent such as, for example, chloroform. The melting agent evaporates but the first and second halves of the polystyrene sphere are melted together along a continuous sealing line, thus maintaining the substantially non-permeable integrity of the hollow inner chamber of the sphere. A plate having a hollow inner chamber may be manufactured in a similar way. In one embodiment, a four-inch by five-inch by one-inch plate was constructed by sandwiching one-half inch plates together. The hollow inner chamber was produced by indenting opposing surfaces with a heated tool, using a method similar to that used for creating hollow spheres. Approximately one inch of solid border was left along the edges of the sheets. The sheets were bonded together using a melting agent such as, for example, chloroform.

Electrostatic tests were conducted to compare the electrostatic energy present on the surfaces of one-inch and three-inch solid and hollow open-cell expanded polystyrene spheres as well as four-inch by five-inch by one-inch solid and hollow open-cell expanded polystyrene plates. The tests were conducted using an electrostatic meter with a sensitivity of 1.0 V/inch. Samples were placed in a microwave field (on non-conducting wood and plastic holders) for one minute. The results of testing on the spheres show, that for both one and three inch spheres, all charge dissipates to a baseline with time (1-2 min) after the microwave field is removed. Dissipation is faster with solid balls compared to hollow, and with dry balls than with balls exposed to an aqueous vapor. Also, distribution of charge on surfaces of solid and hollow balls is highly variable and often polarized, and not relative to air flow direction. The results of the testing on the plates show that all charge dissipates with time (1-2 min) once the microwave field is removed. Dissipation appears to be similar between the solid and hollow plates. Also, distribution of charge on surfaces is highly variable and polarized, mostly along two opposite edges of a plate, and not relative to air flow direction. Accordingly, the tests show that microwave energy generates a static charge on open-cell expanded polystyrene spheres and plates. Hollow spheres and plates retain a greater charge and appear to dissipate the charge slower than their solid counterparts. A summary of the electrostatic energy present on the surfaces of the spheres and plates is shown in Table 1.

TABLE 1
Surface Electrostatic Energy (V/inch)

| | | | | | | 4″ × 5″ × 1″ |
|---|---|---|---|---|---|---|
| | 1″ Solid | 1″ Hollow | 3″ Solid | 3″ Hollow | 4″ × 5″ × 1″ | Hollow |
| Type | Sphere | Sphere | Sphere | Sphere | Solid Plate | Plate |
| Flow Rate | 94 | 94 | 94 | 94 | 185 | 185 |
| (fpm) | | | | | | |
| Control - | .45 | .45 | .77 | .77 | 1.48 | 1.48 |
| Microwave Off | | | | | | |
| No Vapor | | | | | | |
| Microwave On | .51 | .71 | 1.66 | 2.59 | 1.17 | 1.88 |
| No Vapor | | | | | | |
| Control - | .21 | .21 | 1.11 | 1.11 | * | * |
| Microwave Off | | | | | | |
| With Vapor | | | | | | |
| Microwave On | .21 | .57 | 1.11 | 1.47 | * | * |
| With Vapor | | | | | | |

* = Experiment not conducted.

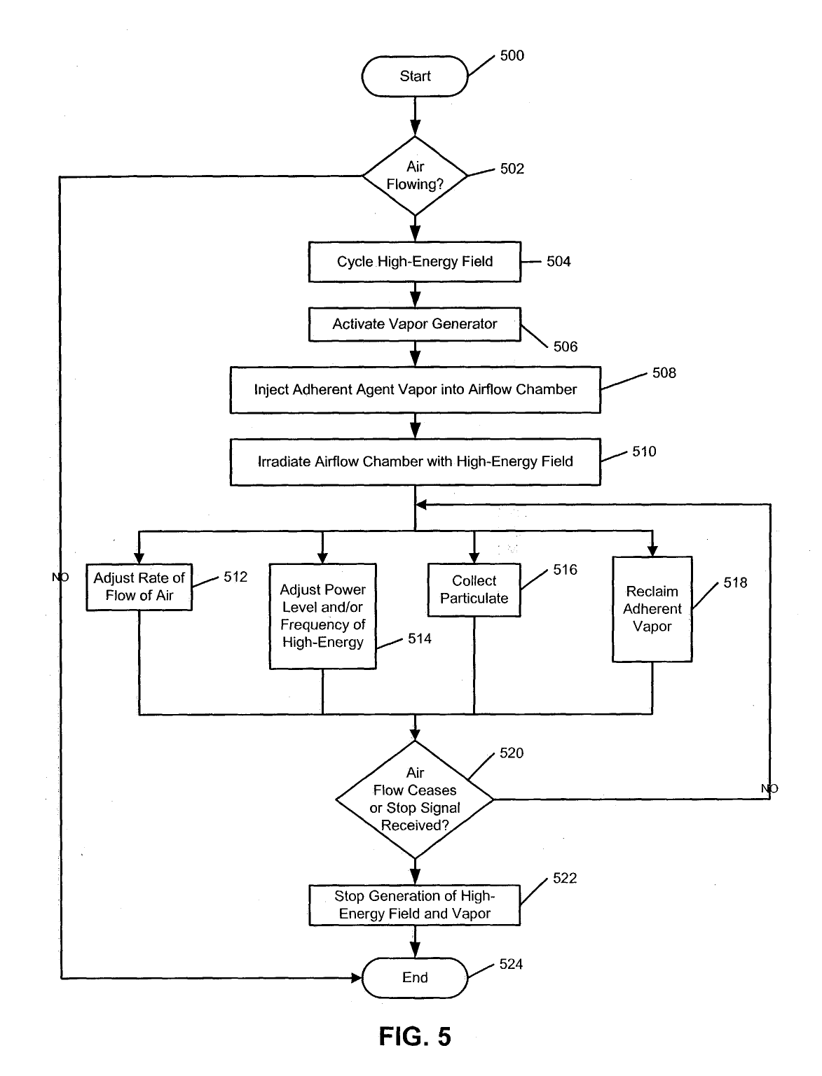

FIG. 5 is a flowchart depicting a method in accordance with an embodiment of the invention. The method may begin at step 500 where an initiation command may be received at a control system. At step 502 a determination may be made as to whether air is flowing through an airflow chamber. If air is flowing through the airflow chamber then, at step 504, a high-energy field, such as a microwave field, may be used to illuminate at least an internal volume of the airflow chamber. In the instance where a microwave field is used, the microwave field may be cycled on and off several times before the system is used. In a preferred embodiment using microwave fields, the system is cycled about three times (approximately one minute each cycle) before use. The high-energy field may induce an electrostatic charge on an external or internal surface of any objects that are present within the airflow chamber. In one embodiment, the objects may be electrically non-conductive and have the property of building-up an electrostatic charge as a result of exposure to a high energy field. In an embodiment using multiple microwave sources, each source, or a subset of the sources, may be cycled.

If air is flowing through the air filtration chamber then, at step 506 a vapor generator may be activated to produce a vapor comprised of an adherent agent. At step 508, the vapor produced by the vapor generator may be introduced into the air flowing through the filtration chamber. The vapor comprised of the adherent agent may have vapor or gas particles that would be large enough to allow pathogenic microbes and other microorganisms present in the air, to be adsorbed by the vaporous adherent agent. The microorganisms in the air may adhere to the adherent agent, such as water droplets, and the microorganism-adherent complex may be carried in the air flowing through the airflow chamber. In one embodiment, the adherent agent is water, however, any aqueous solution that could form a microorganism-adherent complex that may be carried in the air flowing through the airflow chamber would be acceptable. Other non-aqueous adherent agents may also be acceptable and may include Xenon gas, alcohol, or colloidal carbon. The airflow chamber may include objects to increase the tortuosness of the path of air through the airflow chamber. In this case, the objects may also be coated with a film of vaporous adherent agent. The film my increase the likelihood of any free floating microorganisms to attach to the adherent agent and thus to be immobilized on the interior walls of the airflow chamber, or on any objects present in the interior volume of the airflow chamber. Additionally, if the objects were able to hold a static charge, then the likelihood increases that more microorganisms and microorganism-adherent complexes will adhere to the objects.

At step 510, if air is flowing through the airflow chamber, then a high-energy field, such as a microwave field, is used to irradiate the internal volume of the airflow chamber. The high-energy radiation, in combination with the adherent agent, destroys or neutralizes the microorganisms flowing through the system. Such destruction or neutralization could take place when a microorganism, or a microorganism-adherent complex, was freely traveling through the air flow chamber, was electrostatically bonded to an external surface of an electrically non-conductive object present within the air flow chamber, or was otherwise detained within the airflow chamber. Furthermore, microorganisms may have an electrical charge that is opposite in polarity to any electrostatic charge on the external or internal surface of each of the objects that may be present within the air flow chamber, or the walls of the chamber itself. Individual microorganisms may therefore become electrostatically bonded to an external surface of an electrically non-conductive object present within the airflow chamber or a wall of the airflow chamber. The high-energy field, such as the microwave energy field, may then destroy or neutralize the immobilized pathogen.

A microbe may be destroyed or neutralized by absorption of microwaves by virtue of its attachment to a larger water vapor complex. Alternatively, the microbe may be destroyed or neutralized by indirect absorption of heat on the surface of the material, because the water vapor formed a thin layer of liquid water on the surface of the material and this thin layer of water was then super heated. The water film attracted the vapor (to which the microbe was attached), and allowed the microbe to adhere to the surface of the material.

At step 512, the rate of flow of air through the tortuous airflow chamber may be adjusted to ensure up to 100 percent of microorganisms flowing through the tortuous conduit are exposed for a sufficient duration to the high-energy irradiation, to effect the destruction or neutralization of these microorganisms.

At step 514, the power level and/or frequency of the high-energy irradiation may be adjusted to ensure up to 100 percent of microorganisms flowing through the tortuous conduit are exposed to a frequency or power level of high-energy irradiation, to effect the destruction or neutralization of these microorganisms.

At step 516, particulate exiting the sterilization chamber may be captured in a barrier filter.

At step 518, some quantity of adherent vapor exiting the sterilization chamber may be reclaimed (by for example dehumidification or condensation methods) and may be filtered for subsequent reuse in the system.

At step 520, if air ceases to flow through the airflow chamber or if a stop signal is received from the control system, then at step 522 generation of high-energy and generation of adherent agent vapor is stopped and the method may end at step 524. Similarly, if it is determined that air is not flowing at step 502, the method may end at step 524.

Experimental Results

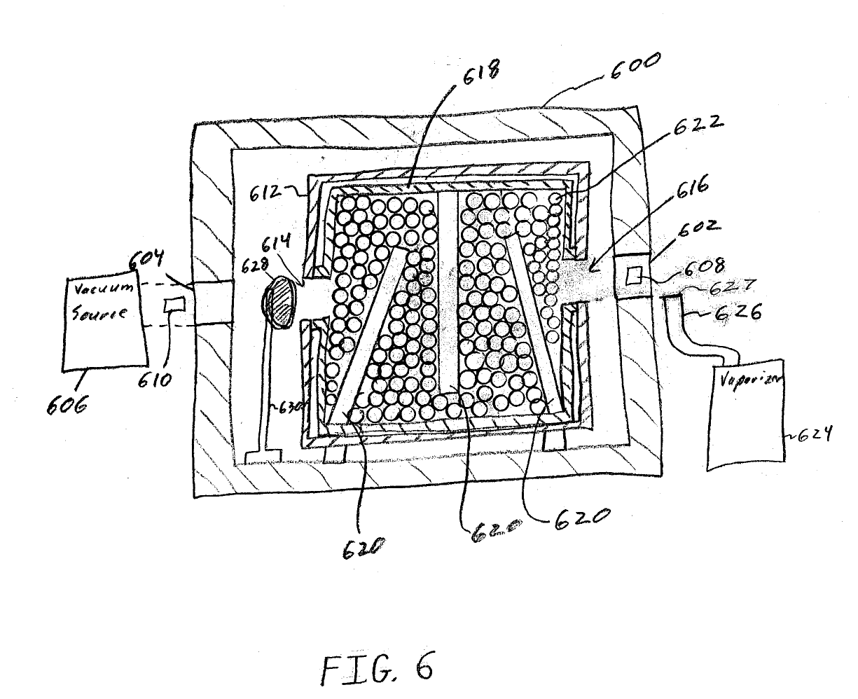

FIG. 6 depicts a cutaway view of a system used to perform laboratory experiments to test the efficacy of air sterilization in accordance with an embodiment of the invention. FIG. 6 illustrates an environmental chamber 600 comprised of an enclosed airtight box with a hinged door. A 2½ inch diameter inlet hole 602 and a 2½ inch diameter outlet hole 604 were cut into the right and left sides of the environmental chamber 600, respectively. The outlet hole 604 was coupled to a vacuum source 606. Alternatively, any other air circulator (not shown) could have been used at either the inlet hole 602 or the outlet hole 604. An anemometer 608 was positioned in the inlet hole 602 to measure the velocity of air entering the air inlet hole 602. A thermometer 610 was positioned at the air outlet hole to measure the temperature of the air flowing from the air outlet hole 604.

A sterilization chamber 612 was positioned within the environmental chamber 600. The sterilization chamber 612 was a commercial 1100 Watt microwave oven that included its magnetron (not shown). The magnetron was a type 2M214 and operated in a continuous wave mode at a frequency of approximately 2,450 MHz. The door of the microwave oven was modified by having an approximately 2½ inch diameter hole cut in its center 614. The rear wall of the microwave oven was modified by also having a 2½ inch diameter hole cut in it 616. The centers of the holes were aligned on approximately the same axial plane. The door of the sterilization chamber 612 could be opened so that an airflow chamber 618 could be placed inside. The environmental chamber 600, sterilization chamber 612, and airflow chamber 618 were designed so that inflow and outflow air holes in each of the three chambers were aligned such that air could be passed through the entire system.

Individual airflow chambers 618 were made of glass, polyethylene, and expanded polystyrene (i.e., Styrofoam®). Whatever the type of material, each airflow chamber 618 included baffles 620 (e.g., interior walls) of the same material. While the outer length of the air flow chamber (i.e., the linear length from air inlet to air outlet hole) was 12 inches, the effective length of travel through the airflow chamber 618, amongst the baffles 620, ranged from approximately 40 inches to approximately 13.5 feet, depending on the internal design of the airflow chamber 618. The internal dimensions of each airflow chamber were approximately 13.5 inches wide×8 inches high×14 inches deep (i.e., distance between inlet and outlet was approximately 14″).

In some experiments, electrically non-conductive objects 622 were placed into the airflow chamber 618 before the airflow chamber 618 was placed within the sterilization chamber 612. The electrically non-conductive objects used were either steam-sealed closed-cell virgin Styrofoam® beads (approximately 3-5 mm in diameter), or open-cell Styrofoam® balls (1 inch in diameter).

Airflow could be applied by either positive or negative pressure (depending on the experiment) to the airflow and sterilization chambers. The vacuum source 606 could be adjusted to obtain a broad range of air speeds as indicated by the anemometer 608.

Water vapor could be added to the air inlet 602 using a vaporizer 624. The vaporizer 624 was an adiabatic piezoelectric type nebulizer with a 25 mm stainless steel disc oscillator operating at 1.7 MHz yielding a 5 ml/min fog achievement. The quantity of vapor could be adjusted as required. The vapor produced by the vaporizer was directed to the air inlet opening 602 by a vaporizer conduit 626, whose opening 627 was adjacent to the air inlet opening 602.

A 100×15 mm sterile plastic petri dish 628 was affixed to a stand 630 such that the petri dish was immediately in front of the air exiting from the sterilization 612 and airflow chambers 618. The pertri dish 628 contained nutrient agar, and was positioned to catch the air exiting the sterilization chamber 612 from air outlet opening 614. In many experiments, the stand 630 could accommodate up to three petri dishes at the same time.

Test microbes could be injected into the inlet using a specially designed injector (not shown) to allow a reproducible quantity of living matter to be injected. The injector was a blind-ended polypropylene tube, 2 mm in diameter and 2 mm long. The bacteria were ground finely between two ground glass plates, and the ground bacteria were then packed into the injector. Reproducible volumes were obtained by packing the ground bacteria to a level even with the tube's open end. Grinding the dried bacteria to approximately the same particle size and packing the ground bacteria at the same pressure insured reproducible bacterial quantities. A smaller tube extended from the side of the injector tube. The smaller tube was fitted to a compressed air supply (not shown) and the living matter explosively sprayed from the injector tube into the inlet 602.

Test organisms were either: 1) dried Brevi bacterium linens, which form a yellow colony on culture; 2) dried Serratia marscesens, which form a red colony on culture; or 3) Penicillium (bread mold spores—probably P. notatum), which form a green colony on culture. Thus, any other nonspecific growth could be detected.

All parts coming in contact with the air flow were cleaned with 100 percent ethyl alcohol and dried in a sterile chamber using ultraviolet light before each use. All sterilization 612 and environmental chamber 600 interiors and exteriors were soaked with spray alcohol and dried before each use. The airflow chamber 618 was soaked in alcohol and dried before each use.

In a multitude of separate experiments, the primary variables tested were: 1) air flow rate (speed); 2) Styrofoamg® packing (and type) versus no Styrofoam® packing inside the air flow chamber; 3) organisms versus no organisms injected; 4) vapor versus no vapor; 5) quantity of vapor; 6) microwave energy applied versus not applied; and 7) types of organisms injected.

General Results

The results of the experiments have shown the following general principles:

1. All three organisms are killed (or survive) proportionally the same;

2. Air flow rate has an inverse and approximately linear effect on kill rate (i.e., rate of destruction and/or neutralization); and

3. Vapor presence has enormous effect on kill rate compared to dry air, yet vapor concentration has little effect on kill rate (yet increased vapor increases the outflow air temperature).

Summary of the Test Results

Summaries of test results are presented in tabular form. Tests to evaluate sterilization were conducted on both solid 1 inch diameter open-cell expanded polystyrene (Styrofoam®) spheres and solid 3-5 mm diameter steam-sealed closed-cell virgin expanded polystyrene beads. Table 2 shows a summary of test results for dried Brevi bacterium linens or dried Serratia marscesens. Results for these two microorganisms were substantially identical. Table 3 shows a summary of test results for Penicillium. The terms “packing material” or “packing type” as used herein refers to either the solid 1 inch spheres or 3-5 mm beads. Results are presented as an approximate “kill rate,” that is a percentage derived from the number of microorganism killed as compared to a control test, where the control test comprised passing microorganisms through the system with no microwave energy, but with all other conditions remaining the same, at the flow rate indicated. The number of microorganisms exiting the system was determined using the impaction-capture method for microbial sampling (described above). Note that the energy requirement to obtain flow through an airflow chamber substantially filled with 3-5 mm beads was approximately three times that required for 1 inch balls, based on the root mean square (rms) power (measured as a function of voltage using an analog voltmeter) required to drive the air handling fan used in the experiments summarized below.

TABLE 2
Brevi Bacterium Linens or Serratia Marscesens
Approximate Kill Rate (%)

| | Packing Type | | | | | |
|---|---|---|---|---|---|---|
| | Balls | Beads | Balls | Beads | Balls | Beads |
| Flow Rate (fpm) | 1600 | 1600 | 680 | 712 | 210 | 292 |
| Microwave On | 45 | 45 | 58 | * | 67 | * |
| No Packing | | | | | | |
| No Vapor | | | | | | |
| Microwave On | 63 | 48 | 81 | 68 | 86 | 78 |
| With Packing | | | | | | |
| No Vapor | | | | | | |
| Microwave On | 98.7 | 91 | 100 | 94 | 100 | 100 |
| With Packing | | | | | | |
| With Vapor | | | | | | |

* = Experiment not conducted.

TABLE 3
Penicillium Approximate Kill Rate (%)

Packing Type

| | Balls | Beads |
|---|---|---|
| Flow Rate (fpm) | 612 | 620 |
| Microwave On | * | * |
| No Packing | | |
| No Vapor | | |
| Microwave On | 90 | 88 |
| With Packing | | |
| No Vapor | | |
| Microwave On | 100 | 100 |
| With Packing | | |
| With Vapor | | |

* = Data contaminated.

Conclusion of Experiments

The experiments demonstrate that even at air flows approaching 8 times the normal air exchange rate of approximately 7 exchanges per hour for a medium sized room of approximately 1900 cubic feet, microwave irradiation in the presence of water vapor can completely destroy and/or neutralize airborne bacteria and spores.

The experiments also demonstrate that destruction and/or neutralization is best achieved if an adherent agent, such as water vapor, is introduced into the microwave irradiation process.

Finally, the experiments demonstrate that the lethality of the system and the system's air flow properties are greatly affected by the material used for the air flow and sterilization chambers, as well as the configuration and composition of the objects used therein.

The material used for the airflow chamber was found to operate maximally if the entire chamber, as well as the packing material (i.e., objects), were composed of open-cell expanded polystyrene (i.e., Styrofoam®). If open-cell expanded polystyrene spheres, approximately one inch in diameter, are used as packing material in the airflow chamber, 100% rate of destruction and/or neutralization of microbes at higher air flow rates than with 3-5 mm diameter spheres is achieved.

Open-cell expanded polystyrene also acquired an electrostatic charge as a result of exposure to a microwave field. The electrostatic charge may have held the microbe-vapor complex to the polymer (expanded polystyrene) with sufficient force to immobilize the complex at higher flow rates. Additionally, open-cell expanded polystyrene has a property of having high surface area. Microscopically, that surface area is many-fold that of the geometric surface area of the material. This affords a higher rate of adsorption of the microbe-carrier complex to the surface of the expanded polystyrene. The higher surface area affords a longer travel for the microbe carrier complex, and affords a larger surface area to attach to (either electrostatically or by water adsorption).

The disclosed embodiments are illustrative of the various ways in which the present invention may be practiced. The present invention may be integrated into new or existing HVAC systems or act as a stand-alone system. The present invention may be used, for example, in homes, hotels, restaurants, office complexes, hospitals, entertainment complexes, aircraft, trains, busses or other vehicles and may be configured as a portable unit for personal use. Other embodiments can be implemented by those skilled in the art without departing from the spirit and scope of the present invention.

What is claimed is:

1. An air sterilization system comprising an adherent agent, a plurality of electrostatically charged non-conductive objects, and high-energy.

2. The system of claim 1, wherein the sterilized air is breathable air.

3. The system of claim 1, wherein the adherent agent is water vapor.

4. The system of claim 1, wherein the adherent agent is a vapor that possesses a net static charge such that a complex comprised of at least one microorganism and at least one particle of the vapor become a dipole.

5. The system of claim 1, wherein at least one of the objects are hollow.

6. The system of claim 1, wherein at least one of the plurality of objects is dissimilar in one of shape and size from the others of the plurality of objects.

7. The system of claim 1, wherein at least one of the objects are spheres.

8. The system of claim 7, wherein at least one of the spheres are hollow.

9. The system of claim 1, wherein at least one of the objects are plates.

10. The system of claim 9, wherein at least one of the plates are hollow.

11. The system of claim 1, wherein the high-energy is one of microwave energy and ultraviolet light energy.

12. An apparatus to sterilize air, comprising:
an enclosed chamber having a inlet opening and an outlet opening and a channel extending therebetween that allows passage of breathable air to be sterilized;
an adherent generator, operationally coupled to the channel, that injects adherent agent into the breathable air to be sterilized; and
at least one high-energy source operatively coupled to the enclosed chamber that irradiates at least a portion of the channel.

13. The apparatus of claim 11, wherein the enclosed chamber is manufactured from a microwave transparent material.

14. The apparatus of claim 11, wherein the enclosed chamber is manufactured from open-cell expanded polystyrene.

15. The apparatus of claim 11, wherein the channel is a tortuous channel.

16. The apparatus of claim 11, wherein the channel comprises at least one bend or curve.

17. The apparatus of claim 11, wherein the channel includes, within its volume, a plurality of electrically non-conductive objects.

18. The apparatus of claim 17, wherein the plurality of electrically non-conductive objects are manufactured from a material that is identical to that used to manufacture the enclosed chamber.

19. The apparatus of claim 17, wherein at least one of the plurality of electrically non-conductive objects is dissimilar in one of shape and size from the others of the plurality of electrically non-conductive objects.

20. The apparatus of claim 17, wherein the plurality of electrically non-conductive objects acquire an electrostatic charge in a microwave energy field.

21. The apparatus of claim 17, wherein at least one of the plurality of electrically non-conductive objects are hollow.

22. The apparatus of claim 17, wherein at least one of the plurality of electrically non-conductive objects are spheres.

23. The apparatus of claim 22, wherein at least one of the spheres are hollow.

24. The apparatus of claim 17, wherein the at least one of the plurality of electrically non-conductive objects are plates.

25. The apparatus of claim 24, wherein at least one of the plates are hollow.

26. The apparatus of claim 20, wherein each of the plurality of electrically non-conductive objects maintains an electrostatic charge with a coating of conductive vapor on an exterior surface of the electrically non-conductive object.

27. The apparatus of claim 17, wherein the adherent generator is one of an atomizer, a mister, a humidifier, and a vaporizer.

28. The apparatus of claim 17, wherein the adherent agent is injected in a vaporous form.

29. The apparatus of claim 17, wherein the adherent agent is water.

30. The apparatus of claim 17, wherein the injection of adherent agent is continuous.

31. The apparatus of claim 17, wherein the high-energy is one of microwave energy and ultraviolet light energy.

32. A method of producing sterilized air, comprising:
flowing air through an enclosed channel, the channel having an inlet opening and an outlet opening that allows passage of the air into and out of the channel, respectively;
injecting adherent agent into the air in the channel; and
irradiating at least a portion of an internal volume of the channel with a high-energy field.

33. The method of claim 32, wherein the sterilized air is breathable air.

34. The method of claim 32, wherein the adherent agent is injected in a vaporous state.

35. The method of claim 32, wherein the adherent agent is injected continuously.

36. The method of claim 32, wherein the high-energy field is one of a microwave field and an ultraviolet light energy field.

37. The method of claim 32, wherein the channel includes a plurality of electrically non-conductive objects that acquire an electrostatic charge as a result of exposure to a microwave field and maintain at least a portion of the electrostatic charge when placed in contact with the adherent agent.

38. The method of claim 32, further comprising:
directing the air flowing out of the channel through an adherent filter; and
separating, in the adherent filter, a quantity of adherent agent from the air flowing out of the channel.

39. A device that acquires a first electrostatic charge when irradiated with a microwave field and maintains a second electrostatic charge, less than the first, when substantially enveloped in a vapor of an electrically conductive aqueous solution, the device comprising: an object having a defined closed exterior surface and a defined closed interior surface, the interior surface juxtaposed to the exterior surface and separated therebetween by a predefined thickness of polymeric material, the interior surface defining a cavity within the object.

40. The device of claim 39, wherein the object is a sphere.

41. The device of claim 39, wherein the object is a plate.

42. The device of claim 39, wherein the polymeric material is open-cell expanded polystyrene.

43. The device of claim 39, wherein the cavity defines a hollow volume.

44. The device of claim 39, wherein the interior surface defines a non-permeable barrier to the vapor.

45. The device of claim 39, further comprising a solid object positioned within and filling the cavity.

46. The device of claim 45, wherein an exterior surface of the solid object defines a non-permeable barrier to the electrically conductive vapor.
